(19) United States
(12) Patent Application Publication
(10) Pub. No.: US 2021/0331989 A1
(43) Pub. Date: Oct. 28, 2021

(54) ON-PURPOSE PROPYLENE PRODUCTION FROM BUTENES

(71) Applicant: Lyondell Chemical Technology, L.P., Houston, TX (US)

(72) Inventors: Lei Zhang, Houston, TX (US); Majid Keyvani, Houston, TX (US); Daniel F. White, Houston, TX (US)

(73) Assignee: Lyondell Chemical Technology, L.P., Houston, TX (US)

(21) Appl. No.: 17/235,418

(22) Filed: Apr. 20, 2021

Related U.S. Application Data
(60) Provisional application No. 63/013,925, filed on Apr. 22, 2020.

(51) Int. Cl. C07C 6/04; C07C 7/00; C07C 7/09; B01J 23/28; B01J 23/30; B01J 21/08

(52) U.S. Cl. CPC C07C 6/04; C07C 7/005; C07C 7/09; B01J 23/28; C07C 2521/08; B01J 21/08; C07C 2523/30; C07C 2523/28; B01J 23/30

(57) ABSTRACT

A low temperature on-purpose propylene production method is described. The method includes autometathesis of butene streams without an initial ethylene feedstock stream using supported autometathesis catalysts that are active at low temperatures. The low temperature allows for liquid phase reactions, which increases the selective production of propylene. The lack of an initial ethylene feedstock stream and low reaction temperature also reduces coking on the autometathesis catalysts, thus extending its lifetime.

### PRIOR RELATED APPLICATIONS

This application claims the benefit of priority to U.S. Provisional Patent Application Nos. 63/013,925, filed on Apr. 22, 2020, which is incorporated herein by reference in its entirety.

### FEDERALLY SPONSORED RESEARCH STATEMENT

Not applicable.

### REFERENCE TO MICROFICHE APPENDIX

Not applicable.

### FIELD OF THE DISCLOSURE

The disclosure relates to processes for producing propylene, particularly to methods of on-purpose production of propylene from C2-C6 olefins.

### BACKGROUND OF THE DISCLOSURE

Propylene is one of the most versatile building blocks in the petrochemical industry in terms of its variety of end-use products and its multitude of production sources. It finds use as a base chemical for a wide variety of applications, including plastics, fuels, and functional derivatives such as acrylonitrile, propylene oxide, cumene/phenol, oxo alcohols, acrylic acid, isopropyl alcohol and oligomers, and the like. By far the most common use of propylene is the production of polypropylene. Polypropylene is the largest volume plastic in the world, greater than low-density, linear low-density, or high-density polyethylene individually. This polymer is mechanically rugged yet flexible, is heat resistant, and is resistant to many chemical solvents like bases and acids. This makes polypropylene ideal for various end-use industries, mainly in packaging and labeling, textiles, plastic parts and reusable containers of various types.

Conventionally, propylene is separated as a byproduct from petrochemical processes. The largest source of propylene is co-production from naphtha or liquefied petroleum gas in ethylene steam crackers. The propylene is a co-product of steam cracking and the quantity produced depends on the nature of feedstock. For heavier feedstocks with larger amounts of propane, butane, and naphtha, the quantity of propylene co-product is about 15%. If the feedstock is light, like ethane, then very little propylene (about 10 times less than naphtha) is produced. This source of propylene, especially in the United States, is diminishing as steam-cracker operators choose to crack ethane because it is an inexpensive component of shale gas.

The second largest amount of propylene (about 30%) comes from refineries as a byproduct from fluidized catalytic cracker (FCC) units that are operated for transportation fuel production. Recently, refiners have been able to increase propylene production in FCC's by optimizing catalyst and operating conditions. However, the potential for production of propylene in existing refinery FCC's is limited by the capacity of the units and the cost to debottleneck to accommodate increased volumes of gas.

Historically, ethylene steam crackers and FCC units have provided almost all of the petrochemical industry's propylene. However, over the past 15 years, the need for key propylene derivatives, such as polypropylene, has grown rapidly and quickly outpaced the need for ethylene derivatives. This increased demand has strained the propylene supply as propylene is still relegated to byproduct status from both steam crackers and FCC units. As a result, there exists a huge gap between market demand and supply of propylene in the world. To address this issue, the petrochemical industry has moved towards “on-purpose propylene” technologies to meet the demands.

Several on-purpose propylene technologies are available, with the most widely used technologies being propane dehydrogenation (PDH), olefin metathesis, and methanol to propylene (MTP). Unfortunately, these technologies have seen limited applicability. PDH, for instance, requires high investment. MTP requires high temperatures that lead to unfavorable propylene selectivity and coking of the active sites on the MTP catalysts. Further, on-purpose propylene production via metathesis is attractive only when the propylene/ethylene pricing spread is significant as it consumes valuable ethylene.

Despite the advances made in on-purpose propylene technologies, there still exists a need for the development of cost-effective technology to selectively produce larger amounts of polymer grade propylene to meet global demand. Even incremental improvements in technology can mean the difference between a cost-effective propylene on-purpose production process, and cost prohibited energy and production losses.

### SUMMARY OF THE DISCLOSURE

The present disclosure provides an improved on-purpose production of polymer grade propylene from butenes. The improved methods rely on autometathesis reactions for a C4 feed stream such as raffinate streams exiting steam crackers and FCC units. Specifically, catalysts that are active at low temperatures without the presence of a significant amount of ethylene are used to facilitate a low temperature butene autometathesis. This results in a thermodynamically-controlled autometathesis reaction that increases the selectively for propylene and increases the conversion of butene to propylene, while reducing coking on the catalyst. The undesired autometathesis product, such as C2 and C4+ olefins, can be recycled back to the reactor for further reactions to increase the amount of polymer grade propylene being produced.

The present methods include any of the following embodiments in any combination(s) of one or more thereof:

A method of producing propylene from a mixed C4 hydrocarbon stream comprising feeding a mixed C4 hydrocarbon stream into an autometathesis reaction zone having a temperature less than 300° C. and a pressure between 0.1 and 5 MPa, wherein the mixed C4 hydrocarbon stream contacts a supported autometathesis catalyst in the autometathesis reaction zone and reacts. A reaction product effluent comprising at least one of ethylene, propylene, C4 hydrocarbons, C5 hydrocarbons, and C6+ hydrocarbons is recovered from the autometathesis reaction zone, wherein it can then be fractionated in a first distillation tower to form an ethylene stream and a C3+ effluent. The C3+ effluent can then be fractionated in a second distillation tower to form a substantially pure, or ultra-pure, propylene stream and a C4-C6+ hydrocarbon stream.

A method of producing propylene from a mixed C4 hydrocarbon stream comprising feeding a mixed C4 hydrocarbon stream into an autometathesis reaction zone, wherein the autometathesis reaction zone has a temperature less than 300° C. and a pressure between 0.1 and 5 MPa, wherein the mixed C4 hydrocarbon stream contacts a supported autometathesis catalyst in the autometathesis reaction zone and reacts. A reaction product effluent comprising at least one of ethylene, propylene, C4 hydrocarbons, C5 hydrocarbons, and C6+ hydrocarbons is recovered from the autometathesis reaction zone, wherein it can then be fractionated in a first distillation tower to form an ethylene stream and a C3+ effluent. The C3+ effluent can then be fractionated in a second distillation tower to form a substantially pure, or ultra-pure, propylene stream and a C4-C6+ hydrocarbon stream. The C4-C6+ hydrocarbon stream can then be combined with the mixed C4 hydrocarbon stream for further reaction in the autometathesis reaction zone to produce more substantially pure and/or ultra-pure propylene.

Any of the above methods, further comprising separating the C4-C6+ hydrocarbon stream into a C4-C5 hydrocarbon stream and a C6+ hydrocarbon stream, wherein the C6+ hydrocarbon stream is purged and the C4-C5 hydrocarbon stream is combined with the mixed C4 hydrocarbon stream for further reaction in the autometathesis reaction zone.

Any of the above methods, further comprising combining the C4-C6+ hydrocarbon stream with the mixed C4 hydrocarbon stream for further reaction in the autometathesis reaction zone.

Any of the above methods, further comprising combining the ethylene stream removed from the first distillation tower with the mixed C4 hydrocarbon stream for further reaction in the autometathesis reaction zone.

Any of the above methods, wherein the supported autometathesis catalyst is W-, Mo-, and Re-based. Alternatively, any of the above methods, wherein the autometathesis catalyst is WO₃, MoO₃, and ReO₃. The autometathesis catalyst is supported by common inorganic solid supports, such as SiO₂ and Al₂O₃.

Any of the above methods, wherein the mixed C4 hydrocarbon stream is a raffinate 1, a raffinate 2, and/or a raffinate 3 stream from a steam cracker or a fluidized catalytic cracker unit.

Any of the above methods, wherein the propylene is at least 95%, 96%, 97%, 98%, 99%, 99.9%, 99.99%, 99.999%, or 99.9999% pure.

A method of producing propylene from a mixed C4 hydrocarbon stream comprising feeding a mixed C4 hydrocarbon stream into an autometathesis reaction zone having a temperature less than 300° C. and a pressure between 0.1 and 5 MPa. The mixed C4 hydrocarbon stream can be a raffinate 1, a raffinate 2, and/or a raffinate 3 stream from a steam cracker or a fluidized catalytic cracker unit. In the autometathesis reaction zone, the mixed C4 hydrocarbon stream contacts a supported autometathesis catalyst and reacts. The autometathesis catalyst can be W-, Mo-, and Re-based, such as WO₃, MoO₃, and ReO₃, each of which is supported by common inorganic solid supports, such as SiO₂ and Al₂O₃. A reaction product effluent comprising at least one of ethylene, propylene, C4 hydrocarbons, C5 hydrocarbons, and C6+ hydrocarbons can be recovered from the autometathesis reaction zone, wherein the effluent can then be fractionated in a first distillation tower to form an ethylene stream and a C3+ effluent. The C3+ effluent can then be fractionated in a second distillation tower to form a substantially pure, or ultra-pure, propylene stream and a C4-C6+ hydrocarbon stream. The ethylene stream and/or the C4-C6+ hydrocarbon stream can be combined with the mixed C4 hydrocarbon stream for further reaction in the autometathesis reaction zone. Alternatively, the C4-C6+ hydrocarbon stream can undergo additional separation processes to remove C6+ hydrocarbons for purging, allowing the remaining C4-C5 hydrocarbons to be combined with the mixed C4 hydrocarbon stream for further reaction in the autometathesis reaction zone.

Any of the above methods, wherein the autometathesis reaction zone also comprises an isomerization catalyst. The isomerization catalyst can be an alkali or alkaline earth-based isomerization catalyst, such as K₂O supported by Al₂O₃ or MgO, or an inorganic solid supported MgO.

Any of the above methods, wherein the supported autometathesis catalyst is a Mo-based catalyst such as MoO₃, and the temperature of the autometathesis reaction zone is between 70 and 150° C.

Any of the above methods, wherein the supported autometathesis catalyst is a W-based catalyst such as WO₃, and the temperature of the autometathesis reaction zone is between 150 and 300° C.

Any of the above methods, wherein the substantially pure propylene stream is polymer grade propylene. Alternative, of the above methods, wherein the ultra-pure propylene stream is polymer grade propylene.

Any of the above methods, wherein the propylene is at least 95%, 96%, 97%, 98%, 99%, 99.9%, 99.99%, 99.999%, or 99.9999% pure.

This summary is provided to introduce a selection of concepts that are further described below in the detailed description. This summary is not intended to identify key or essential features of the claimed subject matter, nor is it intended to be used as an aid in limiting the scope of the claimed subject matter.

### BRIEF DESCRIPTION OF THE DRAWINGS

FIG. 1. A butene autometathesis system according to one embodiment of the disclosed methods.

FIG. 2. Butene conversion for ethylene/butene metathesis and butene-only autometathesis using a supported tungsten-based catalyst.

FIG. 3. Effects of isomerization catalysts on propylene selectivity for butene-only autometathesis with a supported tungsten-based catalyst.

FIG. 4A-B. Catalyst lifetime for reactions with ethylene (FIG. 4A) and without ethylene (FIG. 4B) in the initial metathesis feed, using a supported Mo-catalyst system.

### DEFINITIONS

As used herein, the term “raffinate” refers to a residual stream of olefins obtained after the desired chemicals/material have been removed. In the cracking/crude oil refining process, butene or “C4” raffinate stream refers to the mixed olefin stream recovered from the cracker/fluid catalytic cracking unit. Raffinate 1 refers to the C4 residual olefin stream obtained after separation of butadiene (BD) from the initial C4 raffinate stream. Raffinate 2 refers to the C4 residual olefin stream obtained after separation of both BD and isobutylene from the initial C4 raffinate stream. Raffinate 3 refers to the C4 residual olefin stream obtained after separation of BD, isobutylene, and 1-butene from the initial C4 raffinate stream.

As used herein, the terms “conventional metathesis” and “metathesis” are used interchangeably to refer to the reaction utilizing a C4 hydrocarbon feedstock stream and an ethylene feedstock stream. In contrast, the term “autometathesis” refers to the C4 hydrocarbon feedstock stream reacting in the absence of ethylene as a feedstock. Both metathesis and autometathesis reactions may include additional recycle streams containing undesired reaction products that can undergo further reactions with the feedstock stream(s).

The autometathesis methods described herein uses a feedstock containing both saturated hydrocarbons and olefins, particularly raffinate streams exiting steam crackers and FCC units. While it is possible to enrich the olefin content by processing the raffinate streams with a diverter to remove saturated hydrocarbons, this is not necessary. Further, the extraction efficiency of the conventional butadiene (BD) recovery unit is less than 100%, with approximately greater than 0 to about 0.5 wt % of BD remaining in the raffinate 1, 2, and 3 streams. This small amount of residual BD does not impact the downstream process for the autometathesis catalyst. However, larger amounts of BD must be removed from the autometathesis feedstock. While ethylene is not a feedstock, the autometathesis methods described herein may utilize a recycle stream that includes ethylene, but it is present in such a small amount that it does not affect the supported autometathesis catalyst or the butene conversion.

As used herein, the term “autometathesis catalyst” refers to the compound that ensures the autometathesis reaction takes place, and, for the presently described methods, is supported. The term “isomerization catalyst” is used herein to refer to the compound that is used to rearrange the atoms in a molecule. Both the autometathesis catalyst and the isomerization catalyst can be used simultaneously in the autometathesis reactor to achieve a synergistic effect.

The autometathesis catalysts used for the methods described herein are supported W-, Re-, and Mo-based catalyst that are active at low temperatures. Autometathesis catalyst such as Al₂O₃ or SiO₂ supported WO₃ and MoO₃ may be preferred as they have increased selectively of propylene at the low temperatures used in the presently disclosed methods and are less expensive than Re- and Ru-based catalysts.

The isomerization catalysts can be Mg- or K-based catalysts, and it can also be supported.

As used herein, “catalyst support” refers to a material, usually a solid with a high surface area, to which a catalyst is affixed. The support can be a single inorganic compound or a mixture of inorganic compounds. Exemplary supports can be silica, alumina, zirconia, magnesium or zeolite, including γ-aluminum oxide (γ-Al₂O₃), aluminum oxide (n-Al₂O₃), magnesium oxide (MgO), titanium dioxide (TiO₂), zirconium dioxide (ZrO₂), silicon dioxide (SiO₂), Al₂O₃/SiO₂, Al₂O₃/B₂O₃/SiO₂, and the like. Catalysts affixed to a catalyst support material are referred to as “supported”.

The term “distillation tower” refers to a tower that is capable of separating a liquid mixture into its component parts or fractions by selective boiling and condensation. In a typical distillation, a liquid mixture is heated in the tower wherein the resulting vapor rises up the tower. The vapor condenses on trays inside the tower, and returns to the bottom of the tower, refluxing the rising distillate vapor. The more reflux and/or more trays provided, the better the tower's separation of lower boiling materials from higher boiling materials. Sometimes, a packing material is used in the towers to improve contact between the two phases. For the present methods, the reaction products will need to pass through at least two towers: a de-ethanizer for removal of ethylene overhead and then to a de-propanizer where substantially pure, polymer grade propylene is removed overhead. The bottoms from the de-propanizer can then be recycled, disposed, or sent to a e.g. de-butanizer to separate out C4 from heavier olefins.

The use of the phrase “substantially pure” means a level of purity that is at least 95%. The use of the phrase “ultra-pure” means a level of purity that is at least 99.9999%.

A plus sign (+) is used herein to denote a composition of hydrocarbons with the specified number of carbon atoms plus all heavier components. As an example, a C4+ stream comprises hydrocarbons with 4 carbon atoms plus hydrocarbons having 5 or more carbon atoms.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:
ABBREVIATION TERM
B1 1-butene
B2 2-butenes (including both cis-2-butene and trans-2-butene)
BD butadiene
FCC fluidized catalytic cracker
PDH propane dehydrogenation
MTP Methanol to propylene
WHSV Weight hourly space velocity

### DESCRIPTION OF EMBODIMENTS OF THE DISCLOSURE

The present disclosure provides improved methods and systems for on-purpose production of polymer grade propylene. Specifically, an improved autometathesis process that uses only a butene feedstock from various C4+ fractions of raffinate streams 1-3 is disclosed. This improved method utilizes catalysts that are active at low temperatures such that the autometathesis takes place at lower temperatures, in the liquid phase. Further, the selected catalysts do not require an ethylene feedstock in the initial reaction. This lower reaction temperature and absence of a high ethylene concentration increases the selective production of propylene while suppressing any coking of the catalyst, as compared to the traditional gas phase process. This results in a purer stream of propylene exiting the reaction unit than that exiting the traditional gas phase autometathesis process. A system for use with the improved method is also disclosed.

On-purpose propylene production via metathesis is particularly attractive because it allows for conversion of excess C4+ olefins exiting steam crackers and FCC units to polymer grade propylene per Scheme 1.

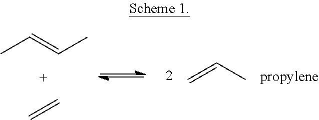

In conventional metathesis methods for producing propylene, a mixed feed of butene and ethylene is reacted with a W-based catalyst in the gas phase. This results in methods that require higher temperatures (e.g. about 250-600° C. for W-based catalyst).

This higher temperature reaction is unfavorable for the thermodynamic equilibrium-controlled propylene selectivity as larger amounts of C5+ olefins are produced instead. To enhance propylene yield, excess C5+ olefinic byproduct undergoes further cracking, thus increasing costs for propylene production.

U.S. Pat. No. 6,777,582 attempts to overcome these issues with autometathesis of a normal butene stream containing 1-butene and 2-butene using a tungsten-based catalyst. To increase propylene selectivity, pentene produced during the autometathesis reaction is recycled back into the autometathesis reactor. The pentene reacts with the 1-butene in the normal butene feedstock to produce more propylene than autometathesis alone, and suppresses the isomerization reaction of 1-butene to 2-butene. Thus, no net pentene is formed in the final reaction product, and the amount of propylene increases.

The present methods overcome the issues in the conventional methods by selecting supported catalysts that are active at low temperatures to convert feed that does not contain ethylene. The lack of ethylene in the initial feed has multiple benefits. It increases the conversion of butenes directly to propylene, and, when the supported catalyst is Mo-based, reduces excess coking caused by ethylene oligomerization at higher pressures. Additionally, the lack of ethylene as an initial reactant is desirable when the supply of ethylene is tight and/or ethylene is expensive due to its own demand.

The currently disclosed methods differ from previous methods because the selected supported autometathesis catalysts do not require ethylene in the initial reactions, and only a small amount of ethylene is produced during the autometathesis reaction. This small amount of ethylene can be recycled back into the autometathesis reactor for further reactions without affecting the catalyst or the butene conversion. Additional reaction products, such as C4-C6+ hydrocarbons, can also be recycled into the autometathesis reactor for further reactions. Schemes 2 and 3 display possible reactions that occur during the butene-only autometathesis process using raffinate streams 1, 2 and 3, wherein the various recycle streams and the butene feedstock generate additional propylene. Alternatively, the C4-C6+ hydrocarbons can be used as feed for other olefin conversion. Thus, the present methods can be a one-pass autometathesis.

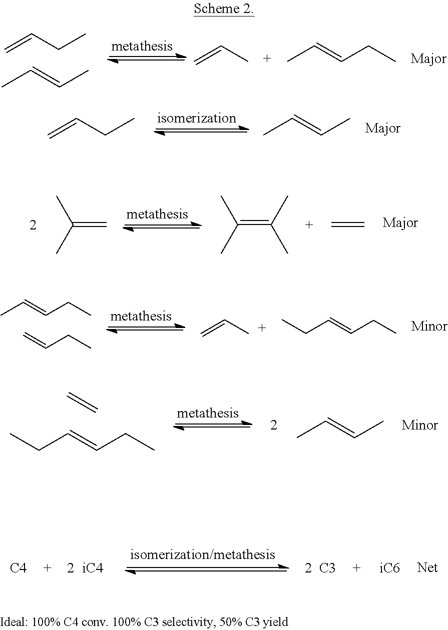

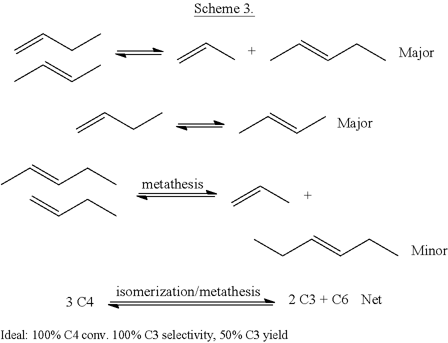

The present methods also utilize lower reaction temperatures. The selected catalyst's low temperatures activity allows the autometathesis reactions to proceed in the liquid phase, which thermodynamically favors production of propylene and reduces coking. This results in a more economically effective production of propylene. This lower temperature, in combination with the lack of an ethylene feedstock, also increases butene conversion and extends the lifetime of the supported autometathesis catalyst.

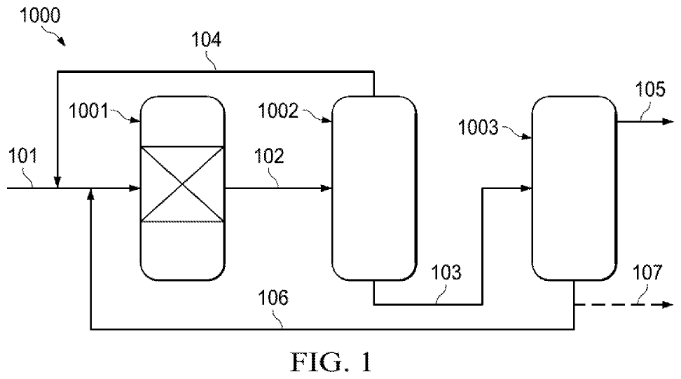

FIG. 1 displays one embodiment 1000 of an on-purpose butene autometathesis system for use with the presently disclosed methods. This system is utilized for the examples described below and is designed to recycle non-preferred and/or undesired autometathesis products back into the reactor for further reactions. However, this embodiment is exemplary only, and the methods can be broadly applied to autometathesis units that dispose of, or utilize, non-preferred reaction products.

For this system 1000, the butene feedstock 101 enters the autometathesis reactor unit 1001, where it reacts with the heterogeneous supported autometathesis catalyst at low temperatures to form a reaction product mixture of C2-C6+ hydrocarbons. The feedstock can include at least one of the C4 raffinate 1, 2 or 3 streams, as well as optional recycle streams 104, 106 containing undesired autometathesis products.

The temperature range for the autometathesis reaction is between about 70 and 300° C. Alternatively, the temperature of the autometathesis reaction is less than 300° C. In yet another alternatively, the temperature is between about 150 and about 250° C. for W-based catalysts, and between about 70 and about 150° C. for Mo-based catalysts. The pressure range for the autometathesis reaction is between 0.1 and 5 MPa. Alternatively, the pressure range is between about 0.5 and 3 MPa or about 2 and 3 MPa. This is lower than the temperature ranges used in previously disclosed autometathesis reactions, which allows the reaction to occur in the liquid phase, not the vapor phase.

The reactor unit 1001 is operated with a fixed bed catalyst and a feed flow rate of about 1 to 10 weight hourly space velocity (WHSV). Autometathesis catalysts that are active at low temperatures (below 300° C.) and on a support material are used in the reactor 1001. This includes oxides of Group VIB and Group VII B metals such as WO₃, MoO₃, and Re₂O₃. Alternatively, the catalyst is W- or Mo-based. Any known support material can be used, including inorganic oxides such as silica, alumina, zirconia, and zeolites.

The resulting reaction product effluent is a mixed C2-C6 hydrocarbon stream 102 that can then be separated according to carbon number groups by technology known in the art. System 1000 displays a series of two distillation towers, wherein the first tower separates out C2 and the second tower separates C3 from C4, C5, and C6+. However, additional distillation towers to separate C4, C5 and C6+ hydrocarbons can also be used in the present system.

In the first distillation tower 1002, the ethylene generated during the autometathesis can be separated from the larger hydrocarbons, removed from the top of the tower 1002 and recycled to the autometathesis tower. While the currently disclosed methods are an improvement because the selected supported autometathesis catalysts do not required ethylene in the initial reactions, the small amount of ethylene produced can still be combined with the feed stream for further metathesis reactions without affecting the supported autometathesis catalyst or creating excess coke on the supported autometathesis catalyst. Alternatively, the ethylene stream 104 can be sent to a C2 splitter and utilized for other processes instead of being recycled (not shown in FIG. 1).

A double bond isomerization catalyst can be used in addition to the supported autometathesis catalyst to suppress formation of heavier, higher carbon hydrocarbons. The double bond isomerization catalyst is also helpful if ethylene is being recycled back into the autometathesis reaction zone. Ethylene reacts with 2-butene (see Scheme 1 above) to form propylene but does not react with 1-butene. An isomerization catalyst can thus be used to isomerize 1-butene to 2-butene to promote utilization of the recycled ethylene with the present methods. The isomerization catalysts are preferably Ca, Mg-, or K-based catalysts, including basic metal oxides, or mixtures thereof. A conventional isomerization catalyst is MgO; however, this isomerization catalyst is known to be sensitive to butadiene poisoning in the C4 raffinate feeds. Therefore, a K-based isomerization catalyst is preferred when using raffinate feeds.

The C3+ stream 103 exits the first distillation tower 1002 from the bottom and is sent to a second distillation tower 1003. The second distillation tower 1003 separates the C3, allowing it to be removed from the top of the tower as a purified C3 stream 105. About 0.2-0.3% of stream 105 is propane, thus this stream is at least a substantially pure propylene stream, if not an ultra-pure propylene stream, that has a polymer grade purity.

The higher carbon olefins (C4+) exit the bottom of the second distillation tower 1003. While embodiment 1000 depicts the recycling of C4-C6+, this is not required by the presently disclosed autometathesis methods.

Per embodiment 1000, the C4-C5 compounds in exit stream 106 can be recycled back to the metathesis reactor unit 1001 while the C6+ compounds can be purged as stream 107. Optionally, the C6+ compounds can be recycling alongside the C4-C5 compounds in stream 106. Purged stream 107 can be disposed of or sent to gasoline blending since stream 107 contains higher octane value aromatics such as benzene in addition to non-aromatic compounds. Alternatively, exit stream 106 can be sent to a third distillation tower to separate the C4 for reuse in the autometathesis reaction while C5 is sent to cracking heaters to produce hydrogenated olefins or sent to gasoline blending.

Using this system and the disclosed methods, autometathesis reaction that can selectively produce propylene, increase butene to propylene conversion, and increase the lifetime of the supported autometathesis catalyst can be achieved. This leads to a more cost-effective process for generating polymer grade propylene.

### EXAMPLES

The following examples are included to demonstrate embodiments of the appended claims using the above described autometathesis system. These examples are intended to be illustrative only, and not to unduly limit the scope of the appended claims. Those of skill in the art should appreciate that many changes can be made in the specific embodiments which are disclosed and still obtain a like or similar result without departing from the spirit and scope of the disclosure herein. In no way should the following examples be read to limit, or to define, the scope of the appended claims.

Butene feedstock: A synthetic raffinate 2 stream with normal butenes was used as a butene feedstock in the following examples. The composition of the raffinate 2 is provided in

TABLE 1
Composition of butene feed used in Example 2

| Component | Mass Fraction (wt. %) |
|---|---|
| 1-butene (B1) | 50 |
| 2-butene (B2) | 50 |

Unless otherwise noted, the flow rates for the butene feedstock is between 1 to 10 WHSV.

Autometathesis catalyst: The following experiments utilized supported WO₃ and MoO₃ as the autometathesis catalysts.

WO₃ catalysts have been used for both metathesis and autometathesis reactions at much higher temperatures than those used here. However, this catalyst was also found to be active at the lower temperatures in the present methods. For the examples below, the support material for the WO₃ autometathesis catalyst was silica.

MoO₃ can be used as an autometathesis catalyst due to its activity at the lower temperatures. For the examples below, the support material for the MoO₃ autometathesis catalyst was alumina.

### Example 1

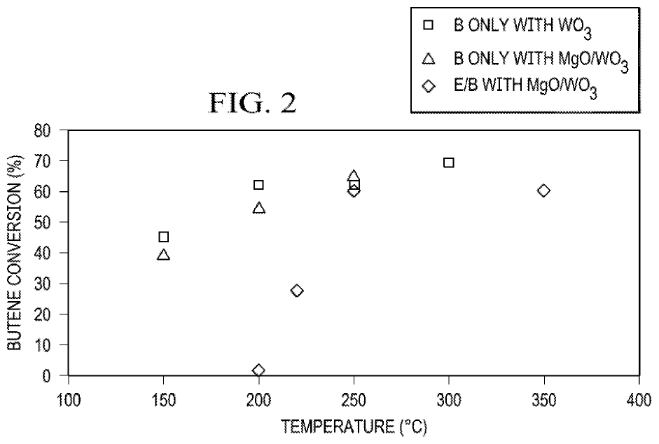

This example addresses the metathesis and autometathesis conversion of butene to propylene at lower reaction temperatures. The conversion of butene to propylene can increase through the combination of lower autometathesis temperatures and removal of an ethylene feedstock stream. This increase in butene conversion, particularly at lower autometathesis reaction temperatures, is illustrated in FIG. 2. FIG. 2 displays the butene conversion for ethylene and butene metathesis (E/B) using a supported WO₃ autometathesis catalyst with a MgO isomerization catalyst, and a butene autometathesis using a supported WO₃ autometathesis catalyst with and without the MgO isomerization catalyst.

In conventional metathesis reactions, temperatures above 300° C. are utilized to produce useful amounts of propylene. Per FIG. 2, temperatures greater than 200° C. are required by the conventional ethylene and butene metathesis to convert the butene to appreciable amounts of propylene. Even at 350° C., only about 60% of the butene was converted.

In contrast, without competing ethylene, the autometathesis of butene takes place at temperatures as low as 150° C. with the supported WO₃ autometathesis catalyst. The conversion rate of 60% was reached at 200° C., over 150 degrees lower than that for the ethylene and butene metathesis (E/B). The reduction in temperature combined with the increasing yield of propylene will reduce the cost associated with the autometathesis process.

The addition of MgO as an isomerization catalyst reduced butene conversion slightly due to increased presence of B2 from isomerization. However, the conversion percentage was still higher than the feedstock that included ethylene.

### Example 2

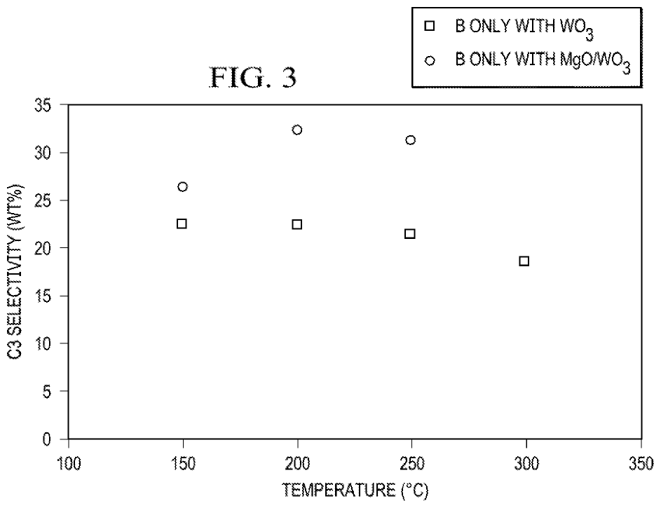

This example addresses the ability to increase the selectivity of propylene with a lower temperature (<300° C.) butene autometathesis. As illustrated in FIG. 3, the supported tungsten-based autometathesis catalyst had a higher propylene selectivity at 150-200° C. before tapering off at higher reaction temperatures. While adding isomerization catalyst such as MgO reduced butene conversion, its presence improved propylene selectivity noticeably, per FIG. 3. This improvement is due to the suppression of the C5+ production. As explained above, higher temperature reactions favor larger amounts of C5+. This reduces the propylene yield but also increases cost due to the need to further treat the heavier hydrocarbons.

### Example 3

Butene conversion and propylene selectivity were evaluated using a supported Mo-based autometathesis catalyst, both with and without K₂O as an isomerization catalyst, with a lower temperature (<300° C.) butene autometathesis. As before, the autometathesis unit in FIG. 1 was used with a synthetic raffinate 2 olefin feedstock. The results of this example are in Table 2.

TABLE 2
Raffinate 2 autometathesis using MoO₃ at 3.10264 MPa

| | Temperature | B1/B | C3 sel. | C5 sel. | B conv. |
|---|---|---|---|---|---|
| Catalyst | (° C.) | (%) | (mol %) | (mol %) | (%) |
| MoO₃ | 70 | 44.79 | 34.28 | 40.48 | 29.79 |
| | 100 | 35.48 | 36.22 | 42.24 | 60.24 |
| | 130 | 30.38 | 34.16 | 42.94 | 65.38 |
| MoO₃/K₂O | 70 | 4.59 | 47.89 | 44.80 | 8.81 |
| | 100 | 6.00 | 49.71 | 39.58 | 35.63 |
| | 130 | 7.55 | 49.33 | 36.66 | 43.55 |

Noticeably lower process temperature favored propylene selectivity in general for the supported Mo-based autometathesis catalyst compared to supported W-based autometathesis catalyst used in Examples 1 and 2. This is due to favorable thermodynamic equilibrium, and means that butene autometathesis using a supported Mo-based catalyst can be performed under high pressure in liquid phase, in contrast to conventional metathesis condition of high temperature. The addition of K₂O also reduced the B1 content (B1/B decreased from greater than 30% to less than 8%) due to isomerization.

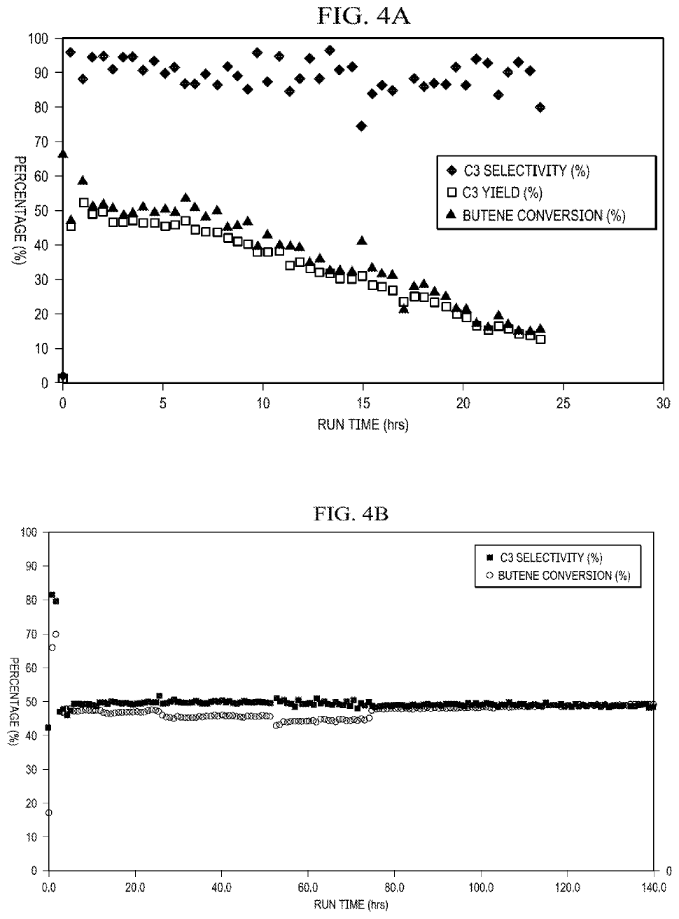

Without a significant amount of competing ethylene, the supported Mo-catalyst was also able to demonstrate longer time on stream without excessive coking caused by ethylene oligomerization at higher pressures. Thus, larger amounts of propylene can be formed before the catalyst has to be replaced or regenerated. FIG. 4A displays the butene conversion and propylene yield for metathesis reactions using ethylene in the initial feed. The conversion rate of butene decreases as the age of the supported catalyst increases to 30 hours. This means that the supported catalyst is no longer efficiently converting the butene.

In contrast, FIG. 4B displays the automethesis reaction using a feed that does not contain ethylene. Even at 140 hours, the supported catalyst is able to efficiently convert butene and shows no evidence of coking at the reaction temperatures used in the presently described methods.

The combination of lower temperature reactions and reduced coking due to the lack of an ethylene feedstock can extend the supported autometathesis catalyst's lifetime by at least more than 20%.

The above examples show that it is possible to increase propylene selectively during a butene autometathesis reaction using raffinate streams and lower temperatures, while also extending the lifetime of the supported autometathesis catalyst by reducing coking. Improving the selectivity of the reaction also reduces costs and equipment needed to further process the undesired reaction production. This ability to selectively produce propylene products while reducing capital costs and energy consumption provides for an efficient on-purpose propylene production to meet the global demands for propylene and its derivative, polypropylene.

The following references are incorporated by reference in their entirety.

U.S. Pat. No. 6,580,009

U.S. Pat. No. 6,646,172

US20170001927

U.S. Pat. No. 8,722,950

U.S. Pat. No. 9,422,209

U.S. Pat. No. 6,777,582

U.S. Pat. No. 8,704,029

1. A method of producing propylene from a mixed C4 hydrocarbon stream comprising:
a) feeding a mixed C4 hydrocarbon stream into an autometathesis reaction zone, said autometathesis reaction zone having a temperature less than 300° C. and a pressure between 0.1 and 5 MPa;
b) contacting said mixed C4 hydrocarbon stream with a supported autometathesis catalyst in said autometathesis reaction zone, wherein the C4s in the hydrocarbon stream react to form a reaction product effluent comprising at least one of ethylene, propylene, C4 hydrocarbons, C5 hydrocarbons, and C6+ hydrocarbons;
c) recovering said reaction product effluent from said autometathesis reaction zone;
d) fractionating said reaction product effluent in a first distillation tower to form an ethylene stream and a C3+ effluent, wherein said ethylene stream is removed from said first distillation tower; and
e) fractionating said C3+ effluent in a second distillation tower to form a substantially pure propylene stream and a C4-C6+ hydrocarbon stream.

2. The method of claim 1, further comprising separating said C4-C6+ hydrocarbon stream into a C4-C5 hydrocarbon stream and a C6+ hydrocarbon stream, wherein said C6+ hydrocarbon stream is purged and said C4-C5 hydrocarbon stream is combined with said mixed C4 hydrocarbon stream for further reaction in the autometathesis reaction zone.

3. The method of claim 1, further comprising combining said C4-C6+ hydrocarbon stream with said mixed C4 hydrocarbon stream for further reaction in the autometathesis reaction zone.

4. The method of claim 1, further comprising combining said ethylene stream with said mixed C4 hydrocarbon stream for further reaction in the autometathesis reaction zone.

5. The method of claim 1, wherein the supported autometathesis catalyst is W-, Mo-, or Re-based.

6. The method of claim 5, wherein the supported autometathesis catalyst is supported WO₃, MoO₃, or ReO₃.

7. The method of claim 1, wherein the autometathesis reaction zone also comprises an isomerization catalyst.

8. The method of claim 7, wherein the isomerization catalyst is an alkali or alkaline earth-based isomerization catalyst.

9. The method of claim 8, wherein the isomerization catalyst is MgO or supported K₂O.

10. The method of claim 1, wherein said supported autometathesis catalyst is supported MoO₃ and the temperature of said autometathesis reaction zone is between 70 and 150° C., or wherein said supported autometathesis catalyst is supported MoO₃ and the temperature of said autometathesis reaction zone is between 70 and 150° C.

11. The method of claim 1, wherein said mixed C4 hydrocarbon stream is a raffinate 1, raffinate 2, and/or raffinate 3 stream from a steam cracker or a fluidized catalytic cracker unit.

12. The method of claim 1, wherein the substantially pure propylene stream is polymer grade propylene.

13. A method of producing propylene from a mixed C4 hydrocarbon stream comprising:
a) feeding a mixed C4 hydrocarbon stream into an autometathesis reaction zone, said autometathesis reaction zone having a temperature less than 300° C. and a pressure of 0.1 and 5 MPa;
b) contacting said C4 hydrocarbon stream with a supported autometathesis catalyst in the autometathesis reaction zone, wherein the C4 in the hydrocarbon stream react to form a reaction product effluent comprising at least one of ethylene, propylene, C4 hydrocarbons, C5 hydrocarbons, and C6+ hydrocarbons;
c) recovering said reaction product effluent from the autometathesis reaction zone;
d) fractionating said reaction product effluent in a first distillation tower to form an ethylene stream and a C3+ effluent, wherein said unreacted ethylene stream is removed from said first distillation tower;
e) fractionating said C3+ effluent in a second distillation tower to form a substantially pure propylene stream and a C4-C6+ hydrocarbon stream; and,
f) combining said C4-C6+ hydrocarbon stream with said mixed C4 hydrocarbon stream for further reaction in the autometathesis reaction zone.

14. The method of claim 13, wherein the autometathesis reaction zone also comprises an isomerization catalyst.

15. The method of claim 14, wherein the isomerization catalyst is an alkali or alkaline earth-based isomerization catalyst.

16. The method of claim 15, wherein the isomerization catalyst is MgO or supported K₂O.

17. The method of claim 13, wherein the supported autometathesis catalyst is W-, Mo-, and Re-based.

18. The method of claim 17, wherein the supported autometathesis catalyst is supported WO₃, MoO₃, and ReO₃.

19. The method of claim 13, wherein the supported autometathesis catalyst is supported MoO₃ and the temperature of said autometathesis reaction zone is between 70 and 150° C., or the metathesis catalyst is supported WO₃ and the temperature of said autometathesis reaction zone is between 150 and 300° C.

20. The method of claim 19, further comprising MgO or supported K₂O in the autometathesis zone.
